(19) United States
(12) Patent Application Publication
(10) Pub. No.: US 2021/0053291 A1
(43) Pub. Date: Feb. 25, 2021

(54) METHOD FOR MANUFACTURING A COMPLEX SUBSTITUTE OBJECT FROM A REAL OBJECT

(71) Applicants: Université de technologie de Compiègne, COMPIÈGNE (FR); Centre National de la Recherche Scientifique, PARIS (FR)

(72) Inventors: Salima BOUVIER, MÉRY-SUR-OISE (FR); Augustin LEREBOURS, COMPIÈGNE (FR); Alain RASSINEUX, LE MEUX (FR); Frédéric MARIN, COMPIÈGNE (FR)

(21) Appl. No.: 16/975,790

(22) PCT Filed: Feb. 26, 2019
(86) PCT No.: PCT/FR2019/050431

§ 371 Date: Oct. 20, 2020

(30) Foreign Application Priority Data
Feb. 26, 2018 (FR) 1851657

(51) Int. Cl. B29C 64/386; A61F 2/42; A61F 2/30; B33Y 50/00; B33Y 80/00; G05B 19/4099

(52) U.S. Cl. CPC B29C 64/386; A61F 2/4261; A61F 2/30942; B33Y 50/00; B33Y 80/00; B29L 2031/7532; A61F 2002/30985; A61F 2002/4276; A61F 2002/4258; A61F 2002/30952; G05B 19/4099

(57) ABSTRACT

The present invention relates to a method for manufacturing a complex substitute object intended to supplement or replace a real object in a given state, potentially a damaged state, in particular a trapeziometacarpal prosthesis intended to replace the trapezoid bone of a human being suffering from rhizarthrosis. The present invention also relates to a trapeziometacarpal prosthesis that can be obtained by the manufacturing method according to the invention.

The present invention relates generally to the manufacture of a complex substitute object intended to complement or replace a real object in a given state, potentially damaged, in particular the manufacture of a trapeziometacarpal prosthesis to replace the trapezium bone of a human being subject to rhizarthrosis.

It is difficult to produce a substitute object such as a prosthesis from a damaged object, above all if it is a part of a human body such as a bone. Nevertheless, if the aim is to produce a prosthesis based on three-dimensional images of a bone which is not damaged, that will not be able to be that of the person for whom the prosthesis is intended. Now, that presents the major drawback that this prosthesis risks not being perfectly suited to that person. The risk of post-implant complications is emphasized by the fact of an instability induced by a mismatch with the environment of the prosthesis. Now, if the bone is very damaged, it is generally difficult and lengthy, even impossible, to use three-dimensional images of that bone as a basis to produce a programming model to drive a numerically-controlled machine tool for the three-dimensional printing of the prosthesis or any other manufacturing means such as machining.

In order to mitigate the abovementioned drawbacks, the applicant has developed a method for manufacturing a complex substitute object having at least one functional zone and intended to complement or replace a real object, said method comprising the following steps:

A. acquisition of a three-dimensional image of said real object taking the form of a point cloud, said three-dimensional image then being digitized;

B. from said digitized three-dimensional image, reconstruction using drawing or computer-assisted design software, of a three-dimensional model of the real object;

C. from said three-dimensional model, programming the driving of a numerically-controlled machine tool to manufacture said complex substitute object;

D. additive manufacturing of said complex object by the numerically-controlled machine tool;

said method being characterized in that the step A is performed on said real object in a specific damaged state with a given deformation, and

in that said method comprises, between the steps A and B, the following substeps:
a′1) definition of invariant topological and morphological parameters of the real object from which a template (in the undamaged state) is defined;
a′2) determination of the functional surfaces (or surfaces of interest) and of the filling surfaces of the real object in the undamaged state;
a′3) definition of the deformation-sensitive topological and morphological parameters of the real object in the damaged state and identification of their variations at its so-called functional surfaces; and

in that the step B further comprises the following substeps:
b1) adaptation of the template to said three-dimensional image defined in the step A, using drawing or CAD software, to obtain a specific model (that is to say a template matched to the damaged state);
b2) on the specific model, precise reconstruction of the functional surfaces in the damaged state from the functional surfaces defined in the step a′2), and approximate reconstruction, of the filling surfaces in the damaged state, from the functional surfaces defined in the step a′2), to obtain a reconstructed three-dimensional image in the damaged state;
b3) precise reconstruction on the reconstructed three-dimensional image in the damaged state, of the functional surfaces in the undamaged state, by modification of the values of the deformation-sensitive topological and morphological parameters defined in the step a′3) so that they correspond to an absence of deformation, to obtain a reconstructed three-dimensional image in the undamaged state;
b4) from the reconstructed three-dimensional image in the undamaged state, extraction of a functional digital file comprising only the zones of interest and a digital filling file comprising the zones other than the zones of interest;
b5) reconstruction from the functional and filling files of a closed volume model of said object to be reconstructed, which is formatted as a neutral file suited to additive manufacturing.

The first step of the method according to the invention is the step A consisting in acquiring a three-dimensional image of said real object which is digitized. This step can be performed in various ways, and notably by three-dimensional digitization (or “3D digitization”) using a 3D scanner. This technique provides three-dimensional synthesis images which do not need to then be segmented.

In the case where the real object that is to be reconstructed or complemented is a part of a human or animal body, the step A will rather be performed in two steps, comprising in particular the acquisition of the three-dimensional images by medical imaging techniques such as by magnetic resonance imaging (generally referred to by the acronym “MRI”), which are then subjected to a subsequent segmentation processing. However, if the images are obtained by 3D scan, they are directly in the form of a point cloud (the segmentation is not therefore necessary).

According to one embodiment of the invention, for example in the case of 3D images by medical imaging and not by 3D scanner, there is, during the step A of acquisition of the three-dimensional image of the real object, a formatting of the image into a neutral format (for example an STL file) compatible with drawing or computer-assisted design software.

Between the steps A and B, the method according to the invention further comprises a substep a′1) of definition of invariant topological and morphological parameters of the real object, from which a template is defined.

Invariant topological and morphological parameters of the real object should be understood to mean, within the meaning of the present invention, parameters which are constantly present from one real object to another, whether or not it is damaged and belonging to the same category or type of object, for example from one trapezium bone to another.

A template is understood to mean, within the meaning of the present invention, an adimensional generic model of an undamaged object (in the sense of not deformed and not damaged by any usage), which is defined by invariant topological and morphological parameters.

The invariant topological and morphological parameters are preferably placed on functional zones.

Functional zones or surfaces of a given object, within the meaning of the present invention, are surfaces of the object having a given function, whether it be of kinematic or other order.

The template can be defined with contiguous or non-contiguous surfaces.

Contiguous surfaces are understood to mean, within the meaning of the present invention, surfaces situated side-by-side and defined by one and the same common curve. That means that the two contiguous surfaces are dependent on one another.

The definition of the common side is similar to the two surfaces, even if they have different functions. Furthermore, there is no free space between the two surfaces.

On the other hand, contiguous surfaces are understood to mean, within the meaning of the present invention, surfaces situated side-by-side but which do not rely on the same curve. That means that the two surfaces are independent of one another and that there is a free space between the two surfaces. It is possible to place several surfaces facing one and the same surface.

The advantage of defining the template by non-contiguous surfaces is to allow the refinement of the most functional zones (that is to say increasing the number of points) having the constraint of ensuring the joining of the different adjacent surfaces.

Between the steps A and B, the method according to the invention further comprises, after the substep a′1), a substep a′2) of determination of the functional surfaces (or surfaces of interest) and of the filling surfaces (of minor importance, comprising the zones other than the zones of interest) of the real object in the undamaged state.

Filling surface is understood to mean, within the meaning of the present invention, any surface which is not functional.

In addition to the substeps a′1 and a′2), the method according to the invention comprises a substep a′3) of determination of the deformation-sensitive topological and morphological parameters of the real object (in the damaged state) and the identification of their variations, at the so-called functional surfaces. This step can be performed from a statistical database of the real geometry of known objects similar to said real object, in order to retrieve an object in the undamaged state, particularly at the so-called functional surfaces.

The step B of the method according to the invention consists in reconstructing, from the possibly segmented three-dimensional image, a three-dimensional model of the real object using drawing or computer-assisted design software.

The step B of the method according to the invention comprises in particular a first substep b1) of adaptation of the template to the digitized three-dimensional image obtained in the step A, using drawing or CAD software, to obtain a specific model, that is to say a template matched to the damaged object.

Moreover, the substep b4) of reconstruction of the functional surfaces in the damaged state consists in modifying, on a reconstructed three-dimensional image in the undamaged state (obtained in the substep b3), the deformation-sensitive morphological parameters, so that they correspond to an absence of deformation on the basis of the information obtained in the substep a′3).

Advantageously, the step B can advantageously comprise, at the end of the step b5), a step b6) of global or localized smoothing of said closed volume model, that can be performed by a surface or volume method. This step is optional but highly recommended because of the small number of points defining the template (the possibility of having sharp edges that can ultimately be harmful for the patient). Moreover, this step is highly recommended if the creation of the closed volume model is performed by voxels.

The method according to the invention is particularly suited to producing complex objects such as arch supports or dental, auditory, or bone prostheses for a human or animal body.

In the case of the production of an implantable prosthesis, it will be possible to use, for the additive manufacturing step D, powders of biocompatible materials such as powders of TA6V, 316L, CrCoMo, or even of ceramic or even of polymer.

In this case, template is understood to mean, within the meaning of the present invention, the adimensional generic model of a part of a human body that is to be replaced or complemented (by a prosthesis or a support), which is that of a healthy subject (person who is not sick), which has not been deformed or undergone degeneration or deformation.

In particular, the method according to the invention is particularly suited to manufacturing a trapeziometacarpal prosthesis as complex substitute object of a trapezium bone of a human being (real object). Such a prosthesis is intended to be implanted in the hand of a patient at the trapeziometacarpal articulation. This implantation is performed in a surgical intervention called prosthetic arthroplasty consisting in replacing, partly or totally, the sick articulation with a prosthesis. This is a therapeutic solution commonly used in the case of arthrosis which is a destruction or degeneration of the articular surfaces.

In the case of the manufacture of a trapeziometacarpal prosthesis as complex substitute object, the zones of interest of the trapezium bone will be the articular surfaces, in other words the surface of articulation with the second metacarpal, the surface of articulation with the scaphoid, the surface of articulation with the trapezoid, and, optionally, the surface defining a groove for the passage of the flexor carpi radialis tendon.

Another subject of the present invention is a trapeziometacarpal prosthesis obtained by the manufacturing method according to the invention as defined specifically for the manufacture of a trapeziometacarpal prosthesis.

Other advantages and particular features of the present invention will emerge from the following description, given as a nonlimiting example and with reference to the attached figures:

FIG. 1 is a plan view of a skeleton of a hand comprising a trapeziometacarpal prosthesis according to the invention;

FIG. 2 is a detail and side view of the prosthesis of FIG. 1;

FIG. 3 is a three-dimensional view of the implantation of the prosthesis of FIG. 1, in which only the first metacarpal and the trapezoid of the skeleton of the hand being represented;

FIG. 4 shows where the trapeziometacarpal articulation is located in the hand of a patient;

FIG. 5 comprises two series of 3D images obtained by MRI then segmentation of the trapeziometacarpal articulation of the hands of two patients suffering from rhizarthrosis, one for the left hand of the two patients (FIG. 5a) and the other for the right hand of these two patients (FIG. 5b);

FIG. 6 shows the topological and morphological parameters that are constantly present from one trapezium bone to another;

FIGS. 7a to 7p show the variations of these topological and morphological parameters of a trapezium bone;

FIGS. 8a to 8c show the changing convex and concave curvatures of the contact surface with the first metacarpal according to the arthrosic state of the articulation;

FIGS. 9a to 9g show the steps A to b6 of digital reconstruction of a trapezium bone that has not undergone deformation linked to rhizarthrosis, in accordance with the method according to the invention, in which the step b5) of reconstruction of a closed volume model of the substitute object to be manufactured is produced by a surface method (called Poisson reconstruction: see FIG. 9f) or by a volume method, called filling by voxels (that is to say digital cubes: see FIG. 9g);

FIG. 10 shows the template of a trapezium bone and all of the points and the curves defining it;

FIG. 11 shows the changes in the smoothing of the closed volume model until the disappearance of the sharp angles is obtained while retaining the integrity of the parameters of a previously defined healthy trapezium bone;

FIG. 12 shows the different elements of the trapeziometacarpal prosthesis to be added (1111) or to be extracted (1111);

FIG. 13 shows the steps C and D of the method according to the invention in the context of the manufacture of a trapeziometacarpal prosthesis, from the creation of the closed volume model of the trapezium bone to be reconstructed up to the additive manufacture and the final smoothing (respectively steps 13d and 13e) culminating in the prosthesis of the trapezium bone, which is finally implanted in the hand of a patient at the trapeziometacarpal articulation (13f);

FIG. 14 shows that the properties of mobility of the hand are retained after a prosthetic arthroplasty operation with the prosthesis of the trapezium bone according to the invention;

FIG. 15 shows the different steps A and B of reconstruction of a cup from a deformed cup.

FIGS. 1 to 15 are described in more detail in the example which follows, given by way of indication, and which illustrates the invention, but without limiting the scope thereof.

### EXAMPLE

### Devices and Instrumentation

Comparator (measurement apparatus): (North and Rutledge, 1983);

Stereophotogrammetry (SPG) on bones of cadavers (25 μm resolution) (Ateshian, 1992; Xu, 1998);

Segmentation of CT scan 0.625×0.3×0.3 mm resolution) (Conconi, 2014; Halilaj, 2014b);

3D laser scan (LS) (Kovler, 2004; Markze, 2012) Micro-CT system on bones of cadavers (0.38 μm resolution);

Image segmentation software: OSIRIX, MATERIALIZE;

Software for formatting segmented images in STL file format: OSIRIX MATERIALIZE;

STL file visualization software: MESCHLAB;

Computer-assisted design software: CATIA V5.

### MATERIALS

### Real Objects

trapezium bones of two male patients suffering from rhizarthrosis, respectively aged 61 years (patient 1) and 66 years (patient 2), whose sick hand is shown in FIGS. 5a and 5b (example 1),
deformed cup (example 2).

### Objects to Be Modeled

trapezium bones of trapeziometacarpal prostheses schematically represented in FIGS. 1 to 3 and shown in the photographs of FIGS. 13d and 13e (example 1),
non-deformed cup (example 2).

### Object to Be Reconstructed

trapezium bone of trapeziometacarpal prostheses schematically represented in FIGS. 1 to 3 and shown in the photographs of FIGS. 13d and 13e.
Material used for the additive manufacture of the prosthesis: CrCoMo, TA6V.

Referring to FIGS. 1 to 3 (relating to the example 1), a trapeziometacarpal prosthesis 1 that can be obtained according to the invention will be described. This trapeziometacarpal prosthesis here comprises a prosthetic trapezium 111, a metacarpal pin 117 and an anchoring screw 113. It is intended to be fitted between the first metacarpal 214 and the trapezoid 212 of a patient. The prosthetic trapezium 111 is anchored on the trapezoid 212 with the anchoring screw 113. In the embodiment illustrated in FIGS. 1 to 3, the metacarpal pin 117 comprises two parts, here added to one another: a base 115 and a head 116.

The object to be reconstructed, namely the cup, is described in example 2 and illustrated in FIG. 15.

### Example 1

### Production of a Trapeziometacarpal Prosthesis According to the Method of the Invention

### 1.1 Acquisition of Segmented Three-Dimensional Images of the Trapezium Bones (Step A of the Method According to the Invention)

The hand of the patient is scanned by MRI or by tomography, then transformed into point clouds (STL format). The technical characteristics of the medical imaging and of the extraction as point cloud (sequences, weighting, contrast) are chosen so as to discretize the cortical part of the bones (FIGS. 5a and 5b).

A digital STL file is extracted, then the identified points defining the template are adapted to the model of the patient in a CAD or mesh editing software. The software MESHLAB, a software of “open source” type, for 3D mesh editing makes it possible, for its part, to visualize the STL files.

When the model is highly arthrosic and damaged, and the template cannot be entirely matched to the patient, the points of unmatched markers are predicted by a healthy bone database. Moreover, the template makes it possible to eliminate the non-anatomical protuberances and roughnesses that are highly present in a bone with an advanced arthrosic state.

### 1.2 Determination of the Topological and Morphological Parameters that are Constantly Present From One Real Object to Another to Obtain a Template (Step A′ of the Method According to the Invention)

FIG. 6 shows the topological and morphological parameters that are constantly present from one trapezium bone to another.

In the biomechanical model of a trapezium bone, three types of marker points are distinguished:
the anatomic marker points (411), defined by experts and corresponding to points that have a biological significance;
the marker pseudo points (412), which are constructed on an object, on a line or between marker points.

Following the reconstruction, common characteristics are revealed (illustrated in FIGS. 7a to 7p):
presence of 4 visible articulation surfaces:
   trapezoid (TPZ) 421,
   metacarpal 1 (M1) 422,
   metacarpal 2 (M2) 423,
   scaphoid (SCP) 424 including 2 planes (SCP and M2), and
the articulation M1 (422) in saddle form.

The choice is therefore focused on highlighting these characteristics.

### 1.3 Determination of the Variable Topological and Morphological Parameters that are Sensitive to Arthrosis (Steps a′1 to a′3 of the Method According to the Invention)

FIGS. 7a to 7p show all of the measurements performed on these marker points in order to know their variability as a function of the arthrosic state. The parameters relating to the contact surface with the first metacarpal are highly sensitive to the arthrosic state (431).

Moreover, FIGS. 8a to 8c show more specifically that the concave (441) and convex (442) curvatures of the contact surface with the first metacarpal vary as a function of the arthrosic state of the articulation. The curvatures are concave are highly pronounced for a healthy subject (4411) and weakly pronounced in the arthrosic state (4412). Conversely, the convex curvatures are highly pronounced in the arthrosic state (4422) and weakly pronounced for a healthy subject. The healthy concave curvatures (4411) decrease along the radioulnar line but are stable for an arthrosic subject (4412). The healthy convex curvatures (4421) increase along the radioulnar line but decrease slightly for an arthrosic subject (4412).

### 1.4 Reconstruction, Using Drawing Software, of a Three-Dimensional Model of a Trapezium Bone not Having Undergone Deformation Linked to the Rhizarthrosis Corresponding to the Trapezium Bones of the Sick Patients (Step B of the Method According to the Invention)

FIGS. 9a to 9f show the different steps of reconstruction of a three-dimensional model of a trapezium bone that has not undergone deformation linked to rhizarthrosis.

More specifically, these figures show in detail:

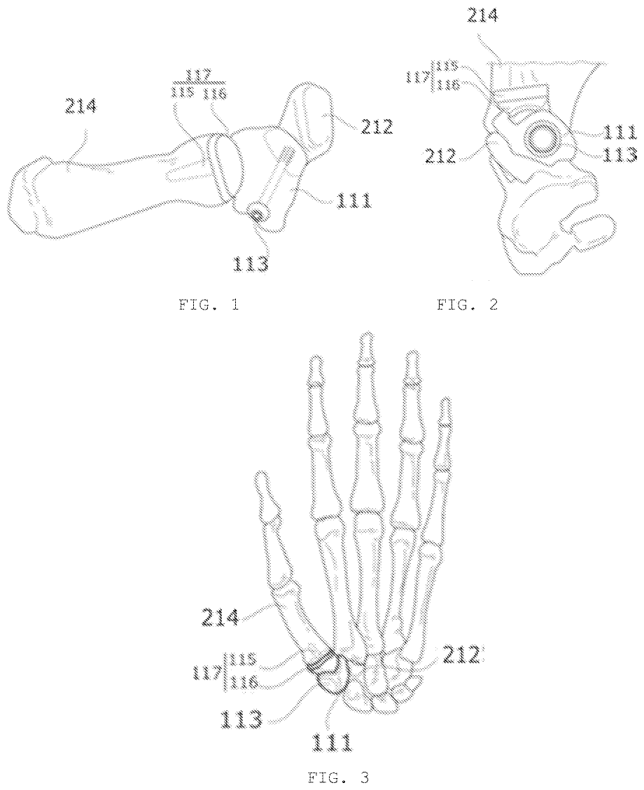

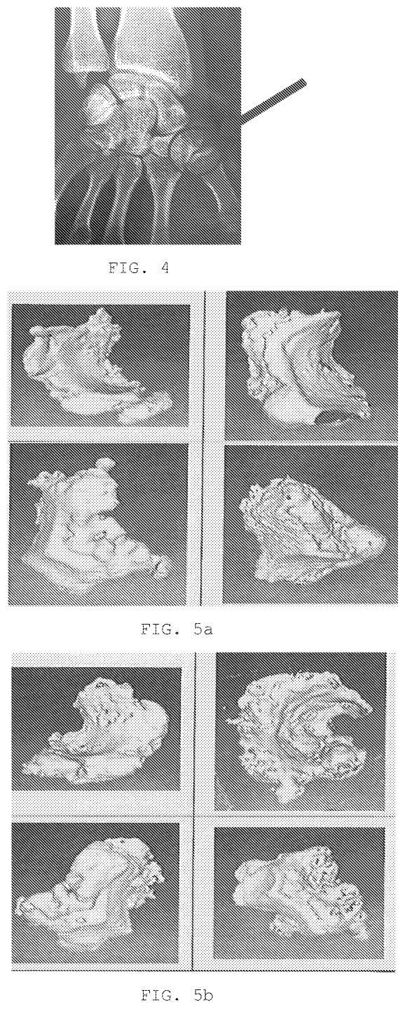

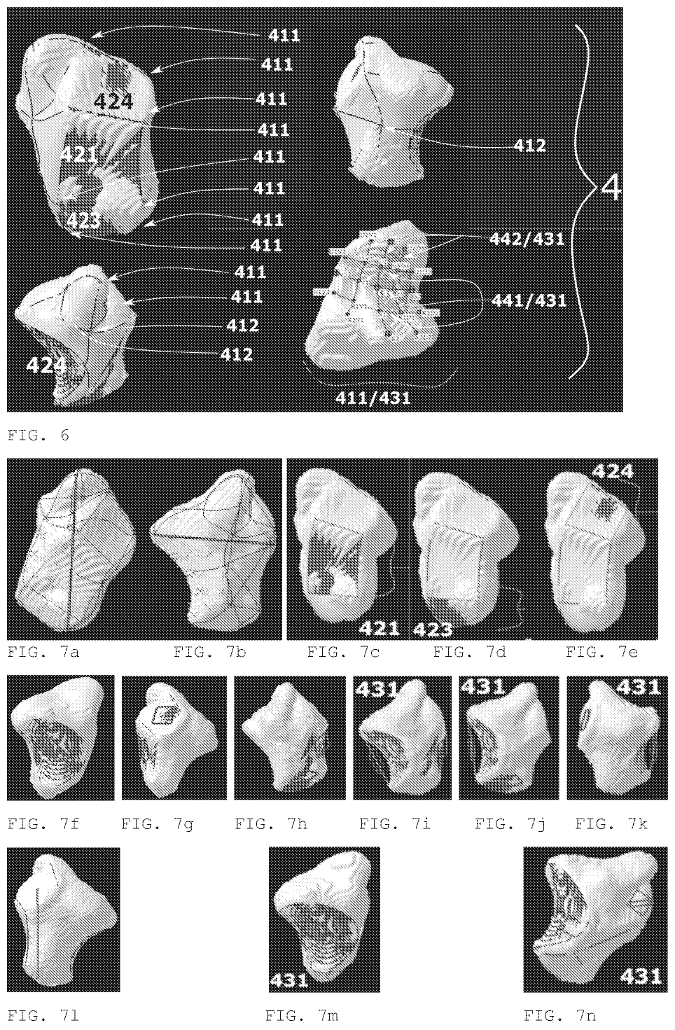

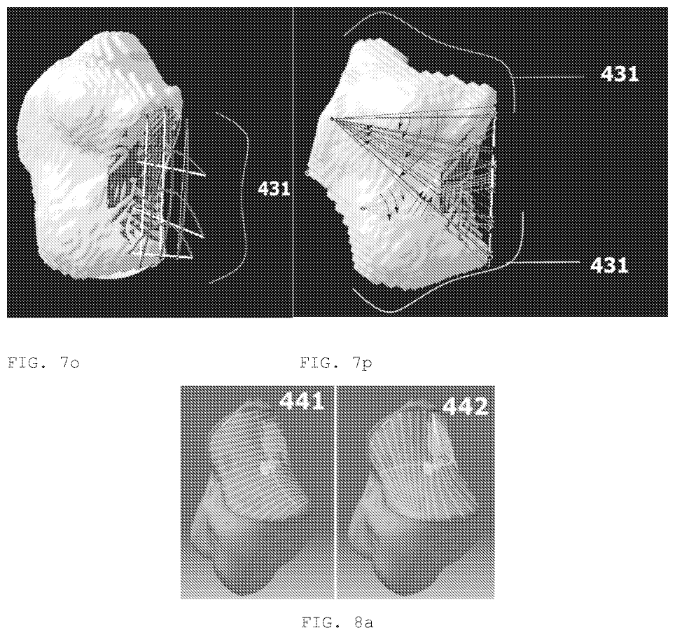

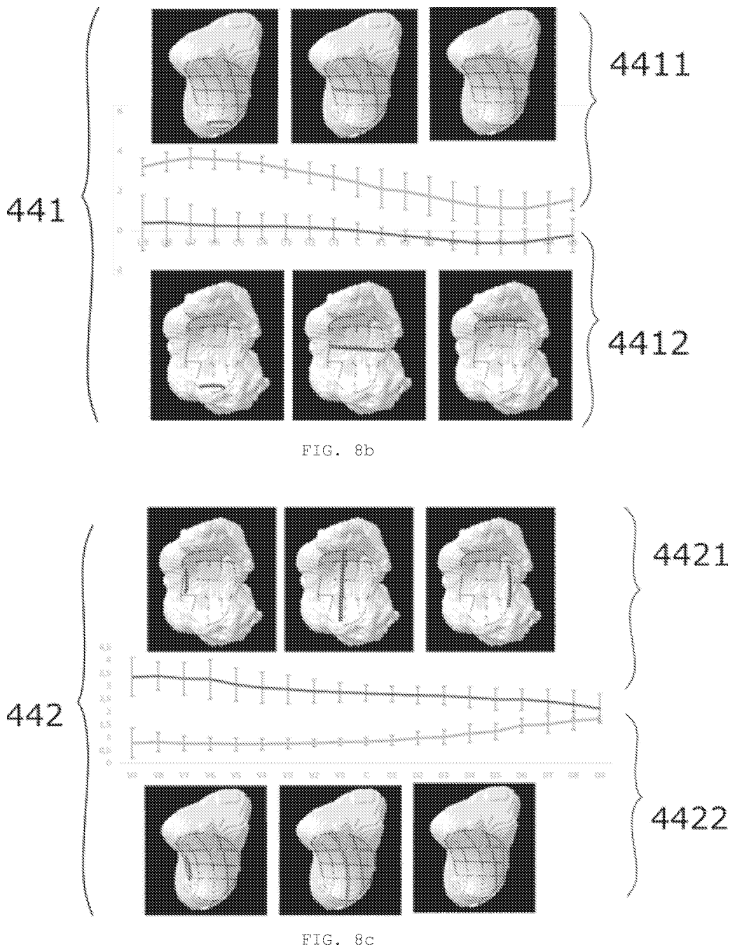

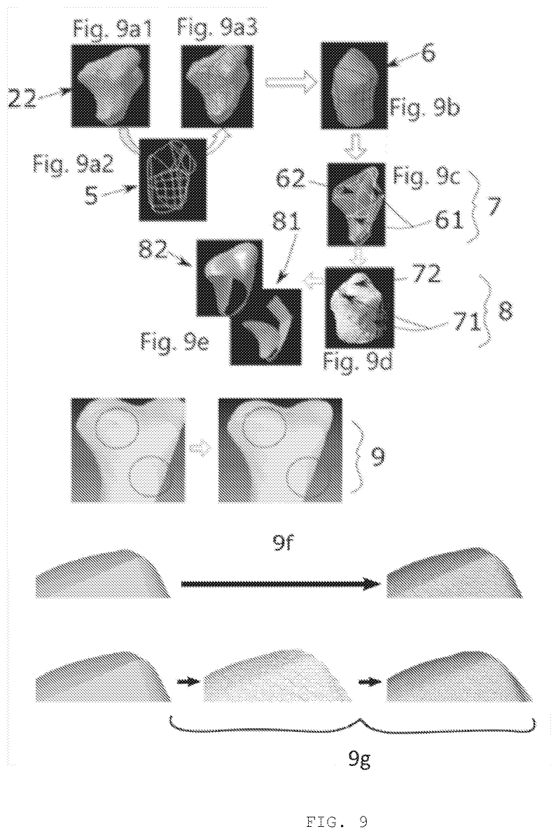

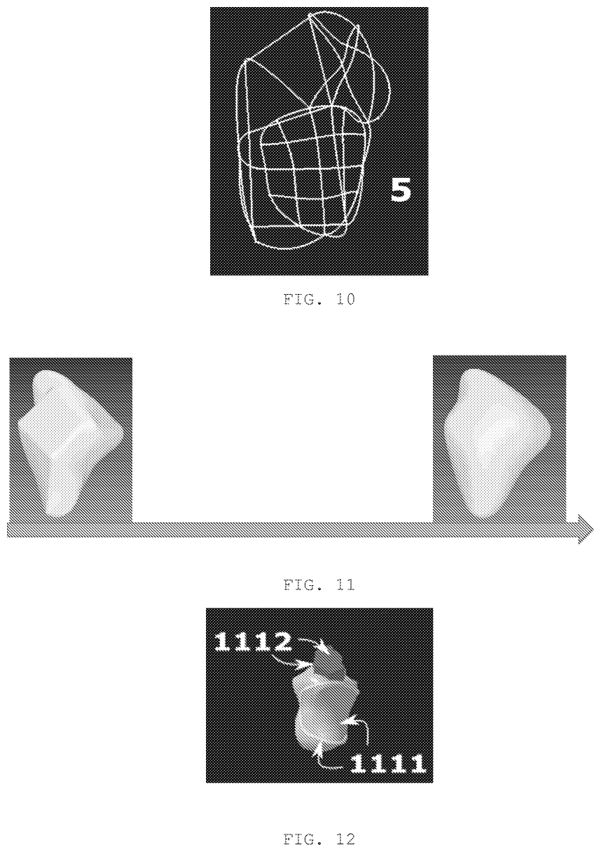

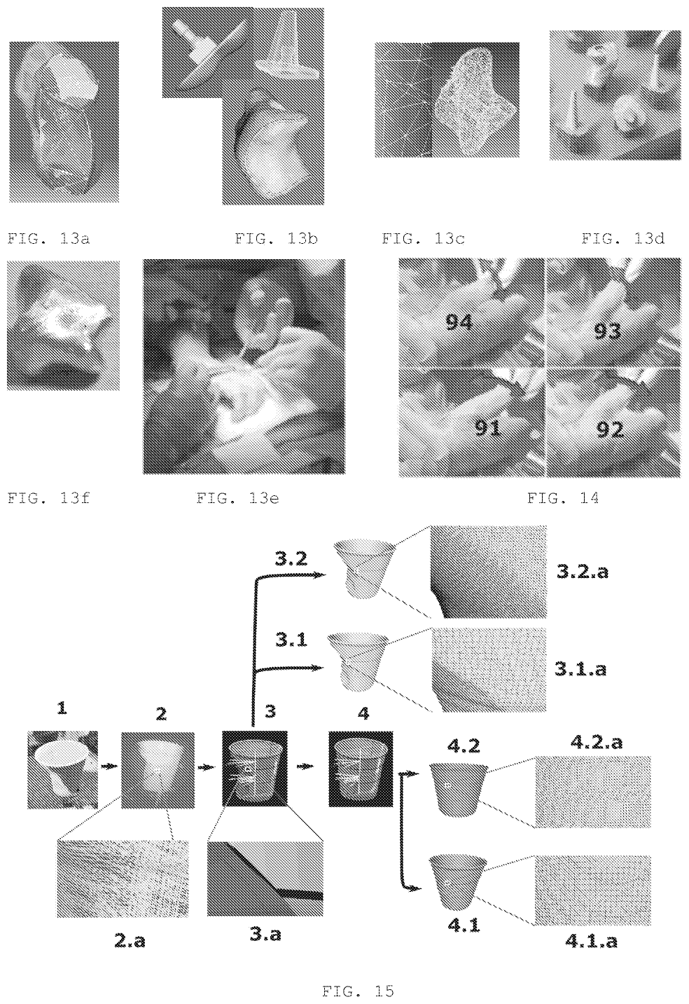

the placement (FIG. 9a3: result of the placement) of the template (of FIG. 9a2) on the 3D digital model of the trapezium bone of the patient (FIG. 9a1),

the identification of the values of the morphological parameters that do not agree with the specifications of a healthy trapezium bone (FIG. 9b),

the identification of the zones of interest and filling zones (FIG. 9c),

the modification of the values of the parameters so that they agree with the specifications of a healthy trapezium bone (FIG. 9d),

the extraction of the zones of interest and filling zones as meshed digital model with sufficient accuracy relative to the tolerances demanded (FIG. 9e),

the reconstruction of a closed 3D model by surface method (Poisson reconstruction: FIG. 9f) or volume method (filling by voxels: FIG. 9g).

### 1.5 Creation by 3D Printing of a Trapezium Bone From the Three-Dimensional Model Obtained in Example 4 (Steps C And D of the Method According to the Invention)

FIG. 14 shows that the properties of mobility of the hand are conserved after a prosthetic arthroplasty operation with the prosthesis of the trapezium bone according to the invention. All of the movements relating to the trapeziometacarpal articulation have been correctly realized, including the retropulsion (91), antipulsion (92), abduction (93), adduction (94) and circumduction movements according to their respective extent.

### Example 2

### Reconstruction, Using Drawing Software, of a Three-Dimensional Model of a Cup That has Not Undergone Deformation From a Deformed Cup (Steps A And B of the Method According to the Invention)

FIG. 15 shows the succession of the steps performed for this purpose from:

1. a deformed cup;

2. creation of a digital point cloud acquired by laser robot arm;

3. creation of a configurable meshed model (in the form of an stl file suited to 3D printing), with unconnected (noncontiguous) zones of the real morphology,
3.1: by Poisson reconstruction, or
3.2: by voxelization and smoothing;

4. creation of a configurable meshed model (in the form of an stl file suited to 3D printing) with unconnected (noncontiguous) zones of the undeformed morphology,
4.1: by Poisson reconstruction, or

4.2: by voxelization and smoothing.

1. A method for manufacturing a complex substitute object having at least one functional zone and intended to complement or replace a real object, said method comprising the following steps:
A. acquisition of a three-dimensional image of said real object taking the form of a point cloud, said three-dimensional image is then digitized;
B. from said digitized three-dimensional image, reconstruction, using drawing or computer-assisted design software, of a three-dimensional model of the real object;
C. from said three-dimensional model, programming of the driving of a numerically-controlled machine tool in order to manufacture said complex substitute object;
D. additive manufacturing of said complex object by the numerically-controlled machine tool;
said method being characterized in that the step A is performed on said real object that is in a damaged state specific to a given deformation, and
in that said method comprises, between the steps A and B, the following substeps:
a′1) definition of invariant topological and morphological parameters of the real object, from which a template is defined;
a′2) determination of the functional surfaces and of the filling surfaces of the real object in the undamaged state;
a′3) determination of the deformation-sensitive topological and morphological parameters of the real object in the damaged state and identification of their variations, at its so-called functional surfaces; and
also characterized in that the step B further comprises the following substeps:
b1) adaptation of the template to said three-dimensional image defined in the step A, using drawing or CAD software, to obtain a specific model;
b2) on said specific model, precise reconstruction of the functional surfaces in the damaged state from the functional surfaces defined in the step a′2) and approximate reconstruction of the filling surfaces in the damaged state from the functional surfaces defined in the step a′2), to obtain a reconstructed three-dimensional image in the damaged state;
b3) precise reconstruction, on said reconstructed three-dimensional image in the damaged state, of the functional surfaces in the undamaged state, by modification of the values of the deformation-sensitive topological and morphological parameters defined in the step a′3) so that they correspond to an absence of deformation, to obtain a reconstructed three-dimensional image in the undamaged state;
b4) from said reconstructed three-dimensional image in the undamaged state, precise definition of the zones of interest of the complex object to be reconstructed, to extract therefrom a functional digital file comprising only said zones of interest and a filling digital file comprising the zones other than the zones of interest;
b5) reconstruction from the functional and filling files of a closed volume model of said object to be reconstructed, which is formatted as a neutral file suited to additive manufacturing.

2. The method as claimed in claim 1, whereby the step B further comprises, at the end of the step b5), a step b6) of global or localized smoothing of said closed volume model.

3. The method as claimed in claim 2, whereby the step b6) is performed by a surface or volume method.

4. The method as claimed in claim 1, whereby, during the step A, there is a formatting of said three-dimensional image into a neutral format compatible with drawing or computer-assisted design software.

5. The method as claimed in claim 1, whereby the complex object to be manufactured is an arch support or a dental, auditory or bone prosthesis, for a human or animal body.

6. The method as claimed in claim 5, whereby the real object is a trapezium bone, for the manufacture of a trapeziometacarpal prosthesis,

7. The method as claimed in claim 4, whereby the zones of interest of the trapezium bone are the articular surfaces with the scaphoid, the trapezoid, the first metacarpal and the second metacarpal.

8. The method as claimed in claim 6, whereby the step D of additive manufacturing is performed based on a powder of biocompatible materials.

9. A trapeziometacarpal prosthesis obtained by the manufacturing method as defined as claimed in claim 6.
